(19) United States
(12) Patent Application Publication
(10) Pub. No.: US 2014/0051104 A1
(43) Pub. Date: Feb. 20, 2014

(54) METHODS FOR PROTEIN PURIFICATION AND ANALYSIS

(71) Applicant: Array Bridge Inc., St. Louis, MO (US)

(72) Inventor: Xing Wang, Wildwood, MO (US)

(21) Appl. No.: 13/946,270

(22) Filed: Jul. 19, 2013

Related U.S. Application Data
(60) Provisional application No. 61/683,247, filed on Aug. 15, 2012.

(51) Int. Cl. C12Q 1/48; C12Q 1/26

(52) U.S. Cl. CPC C12Q 1/485; C12Q 1/26
USPC 435/15; 435/25; 435/287.1

(57) ABSTRACT

Methods to separate complex protein mixtures into individual proteins while maintaining protein function or enzyme activity are disclosed. They are designed to provide high resolution and efficient recovery of the functional proteins. The purified proteins may be analyzed with functional assays including enzymatic activities.

### CROSS-REFERENCES TO RELATED APPLICATIONS

This application claims priority to U.S. Provisional Patent Application Ser. No. 61/683,247, filed Aug. 15, 2012, which is incorporated herein in its entirety.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND

The present disclosure relates to a method for separating complex protein mixtures and, in particular, separating complex protein mixtures while maintaining protein function or enzyme activity.

In the study of protein biochemistry, electrophoresis is widely used to separate mixtures of proteins according to either their electric charge (in no denaturing gel electrophoresis) or the molecular size of the proteins (in SDS-polyacrylamide gel electrophoresis or SDS-PAGE), and to evaluate the purity of the protein preparation. In the one-dimensional gel electrophoresis, one major application of the nondenaturing gel electrophoresis is to detect and locate active proteins directly from the gel, or it could be used to further purify proteins from a mixture in a preparative setting. However, the most useful one dimensional gel electrophoresis is SDS-polyacrylamide gel electrophoresis (SDS-PAGE). In a typical SDS-PAGE, the sample is treated with high concentration of SDS (2%) and in the presence of reducing reagent such as β-mercaptoethanol or Dithiothreitol (DTT). The reducing reagent will allow the dissociation of proteins that are linked by disulfide bounds, and the SDS will bind to the peptide proportional to the mass of the protein. After this treatment, the proteins will migrate in an electric field based on their molecular weight. SDS-PAGE gives much better resolution compared to the nondenaturing gel electrophoresis, and provides a reliable method to estimate the molecular size of the protein.

Even though SDS-PAGE and nondenaturing gel electrophoresis have been widely used in biochemical research, they have several disadvantages. First, in the nondenaturing gel electrophoresis, the resolution is poor, which in turn limits its usage to only partially purified protein preparation if the protein of interest is to be identified positively. Secondly, the in-gel assay to localize the active protein and analyze protein catalytic property is not as efficient as in solution since the proteins are trapped in the polyacrylamide gel. Thirdly, because some proteins are associated to each other in the native form, it is difficult to assess the property of a single protein in the nondenaturing gel electrophoresis. On the other hand, SDS-PAGE provides a better resolution for the proteins, but the electrophoresis procedure is still limited to a partially purified protein preparation if discrete protein bands need to be seen. There are hundreds to thousands of proteins in a single protein preparation from microorganisms, plants or animals, it is almost impossible to separate this number of proteins into discrete bands in the SDS-PAGE. Furthermore, the conditions used to treat the protein samples in SDS-PAGE usually denature the protein in the first place. The sample treatment usually involves the addition of high concentration of reducing reagent and detergent and heated at 100° C. for five minutes. Most of the proteins will be denatured under these conditions, which makes the functional identification of the protein of interest impossible. Therefore, the nondenaturing gel electrophoresis and SDS-PAGE are not suited for the direct purification and functional identification of proteins from a complex protein mixture.

The basic concept of current two-dimensional gel electrophoresis (2-DE) was developed in the 1970s. In a typical 2-DE, proteins are separated in a two dimensional pattern. In the first dimension, proteins are separated according to their isoelectric points. The protein mixture is applied to a pH gradient in an electric field and proteins will migrate according to their electric charge in the pH gradient until to the position where their net charge is zero (isoelectric point). In the second dimension, the proteins are separated according to their molecular weight. Normally a condition similar to the one dimensional SDS-PAGE will be used in this process. Because the separation parameters in the first dimension and second dimension are different from each other, 2-DE gives superior protein separation for highly complex protein samples compared to one-dimensional gel electrophoresis. Since the introduction of the 2-DE, it has been known as the most effective as well as one of the simplest methods of separating most if not all of the proteins from cell crude extract. Over the years, 2-DE has been evolved into a powerful tool for the analysis of complex biological systems especially when the resolution of the 2-DE was improved to more than 10,000 proteins per gel. Another major advancement in 2-DE technology was the introduction of immobilized pH gradient (IPG) gel, which expends the pH range, and the reproducibility of the gel. Currently, proteins separated on the 2-D gel could be identified by microsequencing or mass spectrometry or the combination of both. The online 2-DE database allows direct exchange and comparison of 2-DE data, which serves as a cross reference to the researchers around the world.

The importance of 2-DE could be assessed from several different directions. First, it provides a relatively complete picture of an organism at a defined physiological stage or condition especially for the relatively high to moderate abundant proteins. This is very useful because it has been shown that there is no clear correlation between an organism's gene expression profile and its protein profile. The underline reasons for this observation could be complex but some obvious reasons are mRNA post-transcriptional editing, promoter strength of individual gene and the relative stability of the protein synthesized. Secondly, the elucidations of the protein post-translational modification will generate information that is complementary to the gene transcriptional profile of the organism, and it is mainly proteins that keep organisms operating properly.

The word “proteome” indicates the PROTEins expressed by a genOME or tissue, therefore, proteomics is the study of the proteome of an organism. An organism only has one genome, but could have potentially numerous proteomes because the genome expressed differently under different physiological conditions. In the study of proteome, 2-DE is an important part of the field. Typical proteome study involves the recovery of the protein from a given biological source, display the proteome in a 2-DE, and identify the proteins of interest by mass spectrometry or microsequencing or the combination of both. Given all the advantages of 2-DE, the current 2-DE based proteomics study still fell one step short from the biochemical point of view, i.e. it is unable to monitor the biological activity of the proteins that constitute the proteome. Therefore, the current 2-DE based proteomic study could only generate useful information by comparing two defined physiological status of an organism, for example a diseased tissue vs. a normal tissue, based on the quantity and protein modification, to interpret the biological process or identify the potential drug targets.

### Protein Recovery by Sonication

One of the major challenges for protein characterization after 2-DE is protein recovery from the polyacrylamide gel. This is especially important for the analysis of protein catalytic activity since the in-gel assay is very inefficient. It has been reported that sonication could be used to recover proteins after gel staining. However, no report was known to recover active proteins directly from the 2-D gel using sonication, especially in a high throughput format.

A more widely used method for protein recovery from 2-D gel is electroelution. In the first case, a device called Rotofor Cell is manufactured by Bio-Rad (California, USA) to recover active proteins from isoelectric focusing apparatus. This device uses preparative isoelectric focusing to separate proteins followed by electroelution to recover the separated proteins into different tubes. One of the disadvantages of this device is that it can only be applied to one-dimensional gel, i.e. isoelectric focusing gel, which has a much-decreased resolution when compared to the two-dimensional gel. Another disadvantage of this device is that it uses test tubes to collect the eluted fractions, which makes the resolution of this device very limited. The second device for electroelution is called Whole Gel Eluter also manufactured by Bio-Rad. Again, this device can only be applied to one-dimensional gels, and the resolution of the device is not as good since a very limited member of test tubes are used to collect the potentially hundreds to thousands of proteins separated in a single gel lane. The third device was called Blotelutor Electroelution System that was manufactured by Biometra (Gottingen, Germany). This system uses a semi-dry method to transfer proteins from 2-D gels into a plate that has 576 holes, and the plate is assembled by using a dialysis membrane and a 6 mm thick gel cushion consisting of 12.5% polyacrylamide gel at the bottom of the plate, no data was available on the recovery efficiency of the device. This plate only has effective recovery area of 60%, which means that proteins in the other 40% of the 2-D gel will not be recovered in the process. The resolution of the plate does not provide the opportunity to recover pure proteins from a crude extract because hundreds and even up to thousands of proteins are present in a typical sample. High resolution is in the central part of 2-DE based proteomics research and protein purification. Another issue of the device is the seal of the bottom of the plate with dialysis membrane and polyacrylamide gel; it is not clear whether the proteins will be diffused in this device during protein transfer and recovery, which is an issue if pure proteins need to be purified. The last issue of this device is its inability to adapt to high throughput format, which is very important if it becomes necessary to handle thousands of proteins in a sample and test each of the sample for biological activity as in the case of 2-DE.

Thus, there remains a need for improved methods for protein purification.

### BRIEF SUMMARY OF THE DISCLOSURE

Accordingly, the inventors herein disclose systems and methods for protein purification. They are designed to provide high resolution and efficient recovery of the functional proteins so that they may be analyzed with functional assays including enzymatic activities.

Thus, in various embodiments, the present disclosure provides a method for purifying and characterizing proteins from a mixture comprising: passing the mixture through at least two orthogonal separations under conditions that preserve protein activity; eluting the purified proteins into individual wells of a protein elution plate; and assaying the purified proteins in each well for protein activity.

In various embodiments, the proteins in the mixture may be purified in a first separation according to their isoelectric points. In certain embodiments, this first separation utilizes no reducing agents.

In various embodiments, the proteins in the mixture may purified in a second separation according to their molecular weight.

The second separation, in certain embodiments, may utilize no more than about 2% SDS, no more than about 1% SDS, or no more than about 0.1% SDS.

In various embodiments, the purified proteins may be assayed for NAD reductase activity.

In various embodiments, the purified proteins may be assayed for protein kinase activity.

The method may, in various embodiments, be used to purify and assay protein mixtures obtained from healthy cells. In various embodiments, the method may be used to purify and assay protein mixtures obtained from diseased cells.

In another embodiment, the method may further involve quantifying the purified proteins.

In various embodiments, method may further involve identifying the purified proteins. In certain embodiments, the purified proteins may be identified by protein microsequencing. In certain embodiments, the purified proteins may be identified by mass spectrometry.

The present disclosure provides a system for purifying and characterizing proteins from a mixture comprising: a separating apparatus that performs at least two orthogonal separations under conditions that preserve protein activity; and a protein elution plate.

In various embodiments, the separating apparatus comprises an IPG (immobilized pH gradient) strip.

In various embodiments, the separating apparatus further comprises a polyacrylamide electrophoresis gel.

In various embodiments, the system utilizes no reducing agents.

In various embodiments, the system utilizes no more than about 2% SDS, no more than about 1% SDS, or no more than about 0.1% SDS.

In certain embodiments, the protein elution plate has a plurality of receiving wells. In a particular embodiment, the protein elution plate has 1,536 receiving wells.

The system according to claim 16, wherein the protein elution plate comprises polypropylene.

In certain embodiments, the protein elution plate further comprises a semi-permeable membrane.

In certain embodiments, the semi-permeable membrane is attached to the protein elution plate through a gel.

In certain embodiments, the semi-permeable membrane comprises polyethersulfone or polyamide polymer.

### BRIEF DESCRIPTION OF THE SEVERAL VIEWS OF THE DRAWING(S)

The details of the present disclosure, both as to its structure and operation, may be gleaned in part by study of the accompanying drawings, in which like reference numerals refer to like parts, and in which:

FIG. 1 is a diagram of the Protein Elution Plate (PEP) design (dimensions are in millimeters); and

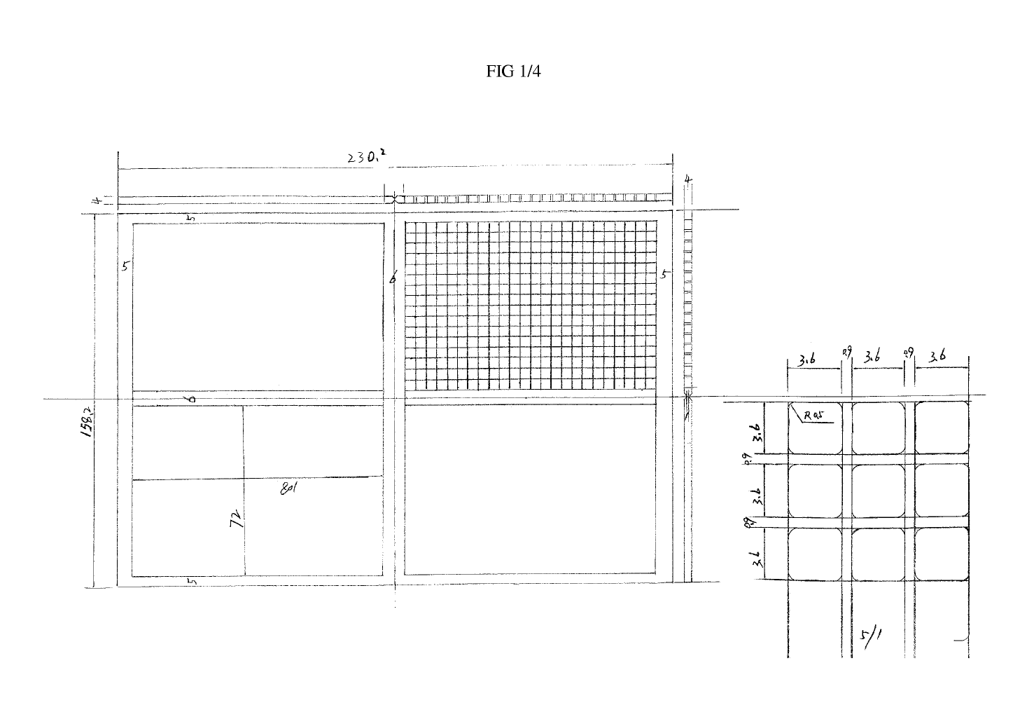

FIG. 2 is an assay procedure diagram for the use of PEP to recover and analyze functional proteins separated with two-dimensional gel electrophoresis; and

FIG. 3 illustrates a transfer of separated proteins from a 2-D Gel to a PEP Recovery Plate; and

FIG. 4 illustrates that protein recovered from individual wells of the PEP is relatively pure.

### DETAILED DESCRIPTION OF THE DISCLOSURE

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “isoelectric point” refers to the point at which a molecule or compound, which can exist in forms bearing either negative and/or positive charges, is electrically balanced, such that the net charge on the molecule or compound is zero.

The term “protein” refers to any chain of amino acids, regardless of length or post-translational modification. Proteins can exist as monomers or multimers, comprising two or more assembled polypeptide chains, fragments of proteins, polypeptides, oligopeptides, or peptides.

The term “purified protein or peptide” as used herein, is intended to refer to a composition, isolatable from other components, wherein the protein or peptide is purified to any degree relative to its naturally obtainable state. A purified protein or peptide therefore also refers to a protein or peptide, free from the environment in which it may naturally occur. A purified protein or peptide is said to “preserve its activity”, if the biological activity of the protein, such as an enzyme, at a given time, is within about 10% (within the errors of the assay) of the biological activity exhibited by the protein in a mixture. According to the teaching of this disclosure, biological activity may be persevered by using minimal or no reducing agents or SDS during separation.

As used herein, the term “active protein”, “biologically active protein,” “bioactive protein,” “biologically active protein fragment” or “bioactive protein fragment” is any polypeptide or fragment thereof derived from a mixture according to the teaching of this disclosure that has biological activity, e.g., enzymatic activity, etc. Thus, the term “bioactive protein” refers to a protein having biological activity.

The term “reducing agent” refers to agents used to reduce the disulfide bonds in proteins. Commonly used reducing reagents are β-mercaptoethanol, dithiothreitol (DTT), dithioerythritol (DTE), and glutathione.

The term “SDS” refers to sodium dodecyl sulfate, which is also known as sodium laurilsulfate or sodium lauryl sulfate (SLS). It is an organic compound with the formula CH₃(CH₂)₁₁OSO₃Na. In sufficient concentrations, this compound disrupts non-covalent bonds in proteins, denaturing them, and causing the molecules to lose their native shape and activity.

The term “protein elution plate” or “PEP” refers to an elution plate comprising a plurality of wells. In certain embodiments the number of wells ranges from about 200 to about 2000; in certain instances 1,536 96. The PEP is configured to receive purified proteins eluting from an electrophoresis gel.

The term “polypropylene” refers to any polymer comprising propylene polymerization units, regardless of whether the polymer is a homopolymer or a copolymer, and further includes blends of such homopolymers and copolymers.

The term “semi-permeable membrane” refers to a membrane that displays different permeabilities for different species of molecules, and therefore, may be used in the separation of ions and molecules having different permeabilities across the membrane.

The term “gel” refers to a network of either entangled or cross-linked polymers swollen by solvent. The term is also used to describe an aggregated system of colloidal particles that forms a continuous network.

The term “polyethersulfone” refers to a polymer formed from condensation of a diphenol (such as bisphenol-A or hydroquinone) and bis(4-chlorophenyl)sulfone.

The term “polyamide” refers to a polymer in which amide linkages (—C(O)NH—) occur along the molecular chain.

The phrase “kinase activity” refers to the ability of an enzyme to catalyze the transfer of a phosphate from one molecule to another. Purified proteins that display protein kinase activity are understood to contain enzymes capable of transferring a phosphate from one molecule to another.

The phrase “NAD⁺ reductase activity” refers to the ability of an enzyme to catalyze the reduction of NAD⁺ (nicotinamide adenine dinucleotide) to its reduced form, NADH. Purified proteins that display NAD⁺ reductase activity are understood to contain enzymes capable of reducing NAD⁺.

The phrase “Protein sequencing” refers to techniques to determine the amino acid sequence of a protein. The phrase “Protein microsequencing” refers to techniques for determining the amino acid sequence of very small amounts of protein.

### Methods

Certain embodiments as disclosed herein provide methods for separating complex protein mixtures and, in particular, separating complex protein mixtures while maintaining protein function or enzyme activity.

Two conditions were developed to maintain the protein function including enzymatic activities. First, no reducing reagent was used in the Isoelectric Focusing step, keeping the disulfide bonds in the proteins intact. Second, a reduced SDS concentration was used in the SDS-PAGE (from 2% to 0.1%), again maintaining protein function (there are many examples in literature indicating that enzymes are active at low levels of SDS). Furthermore, a high resolution Protein Elution Plate was designed that contains 1,536 wells in a microplate-compatible format. This Protein Elution Plate is made of polypropylene or any other plastic or synthesized material. At one side of the Protein Elution Plate, a semi-permeable membrane is attached, this could be made of polyethersulfone or polyamide or other material that has a certain molecular cut-off. The membrane is attached to the Protein Elution Plate through a gel. In one case, Super 77 gel spray from 3M was used for the attachment of the membrane, other gels can also be used for the production of the plate. Since a typical biological sample contains less than 2,000 major proteins, theoretically, each well in the Protein Elution Plate could contain just one protein species, which will allow for the one-step purification of proteins and the assignment of the protein function (enzymatic activity) to the protein identified through mass spectrometry or microsequencing. Through this approach, a systematic measurement of enzymatic activities may be made and a 2-D enzymatic activity landscape may be developed (see the examples below). This will provide systematic knowledge of disease development, and a possible new way for drug target identification.

Thus, in various embodiments, the present disclosure provides a method for purifying and characterizing proteins from a mixture comprising: passing the mixture through at least two orthogonal separations under conditions that preserve protein activity; eluting the purified proteins into individual wells of a protein elution plate; and assaying the purified proteins in each well for protein activity.

In various embodiments, the proteins in the mixture may be purified in a first separation according to their isoelectric points. In certain embodiments, this first separation utilizes no reducing agents.

In various embodiments, the proteins in the mixture may be purified in a second separation according to their molecular weight.

The second separation, in certain embodiments, may utilize no more than about 2% SDS, no more than about 1% SDS, or no more than about 0.1% SDS.

In various embodiments, the purified proteins may be assayed for NAD+ reductase activity.

In various embodiments, the purified proteins may be assayed for protein kinase activity.

The method may, in various embodiments, be used to purify and assay protein mixtures obtained from healthy cells. In various embodiments, the method may be used to purify and assay protein mixtures obtained from diseased cells.

In another embodiment, the method may further involve quantifying the purified proteins.

In various embodiments, method may further involve identifying the purified proteins. In certain embodiments, the purified proteins may be identified by protein microsequencing. In certain embodiments, the purified proteins may be identified by mass spectrometry.

The present disclosure provides a system for purifying and characterizing proteins from a mixture comprising: a separating apparatus that performs at least two orthogonal separations under conditions that preserve protein activity; and a protein elution plate.

In various embodiments, the separating apparatus comprises an IPG (immobilized pH gradient) strip.

In various embodiments, the separating apparatus further comprises a polyacrylamide electrophoresis gel.

In various embodiments, the system utilizes no reducing agents.

In various embodiments, the system utilizes no more than about 2% SDS, no more than about 1% SDS, or no more than about 0.1% SDS.

In certain embodiments, the protein elution plate has a plurality of receiving wells. In a particular embodiment, the protein elution plate has 1,536 receiving wells.

The system according to claim 16, wherein the protein elution plate comprises polypropylene.

In certain embodiments, the protein elution plate further comprises a semi-permeable membrane.

In certain embodiments, the semi-permeable membrane is attached to the protein elution plate through a gel.

In certain embodiments, the semi-permeable membrane comprises polyethersulfone or polyamide polymer.

After reading this description, it will become apparent to one skilled in the art how to implement the disclosure in various alternative embodiments and alternative applications. However, although various embodiments of the present disclosure will be described herein, it is understood that these embodiments are presented by way of example only, and not limitation. As such, this detailed description of various alternative embodiments should not be construed to limit the scope or breadth of the present disclosure as set forth in the appended claims.

### EXAMPLES

### Analysis of NAD(+) Reductase and Protein Kinase from Lung Benign and Cancer Cell Lines

The experiment was divided into 6 steps: 1.) protein preparations from both benign and cancer cell lines were prepared from cell culture, and a BCA method was used to quantify the protein concentration. 2.) 400 μg/each of the proteins from the lung benign and cancer cells were loaded onto an IPG (immobilized pH gradient) strip respectively and separated by Isoelectric Focusing (IEF). 3.) The proteins separated by IEF were further separated by a modified second dimensional polyacrylamide gel electrophoresis, which will display the separated proteins in a two-dimensional pattern and still keep the enzyme activities of the proteins active. 4.) The proteins in the gel were eluted into a specially designed plate, called Protein Elution Plate, which has 1,536 wells. 5.) The samples from the Protein Elution Plate were transferred to four 384-well microplates. 6.) Enzyme assays were performed for NAD+ Reductase and Protein Kinase activities separately, and the data was collected and analyzed with Microsoft Excel.

### Testing the Protein Transfer Efficiency of the Protein Elution Plate

Seed proteins (400 μg) were loaded onto an IPG strip and separated by isoelectric focusing (IEF), and further separated by second orthogonal gel electrophoresis. An aspect of the study was to retain the enzymatic activity during the separation and protein transfer. In a typical 2-D gel electrophoresis, reducing reagents such as β-mercaptoethanol or Dithiothreitol (DTT) are used to reduce the disulfide bonds to improve the separation efficiency in accordance with a high concentration of urea (normally 8 M). Typically, these reagents are added to denature the proteins so that the proteins can be separated more easily. However, in our experiment, the purpose is to separate the proteins efficiently while retaining the enzymatic activities. Therefore, modifications were made for the 2-D gel electrophoresis process. First, no reducing reagent was added in the IEF gel to keep the disulfide bonds in the protein. Second, a much-reduced SDS concentration is used for the IPG strip incubation. Instead of the typical 2% SDS with reducing reagent, the SDS concentration was reduced 20-folds to 0.1%. In several preliminary experiments, it was shown that at this concentration, the proteins still exhibited good separation and efficiency, and, the testing enzyme (Horse Radish Peroxidase) was still active.

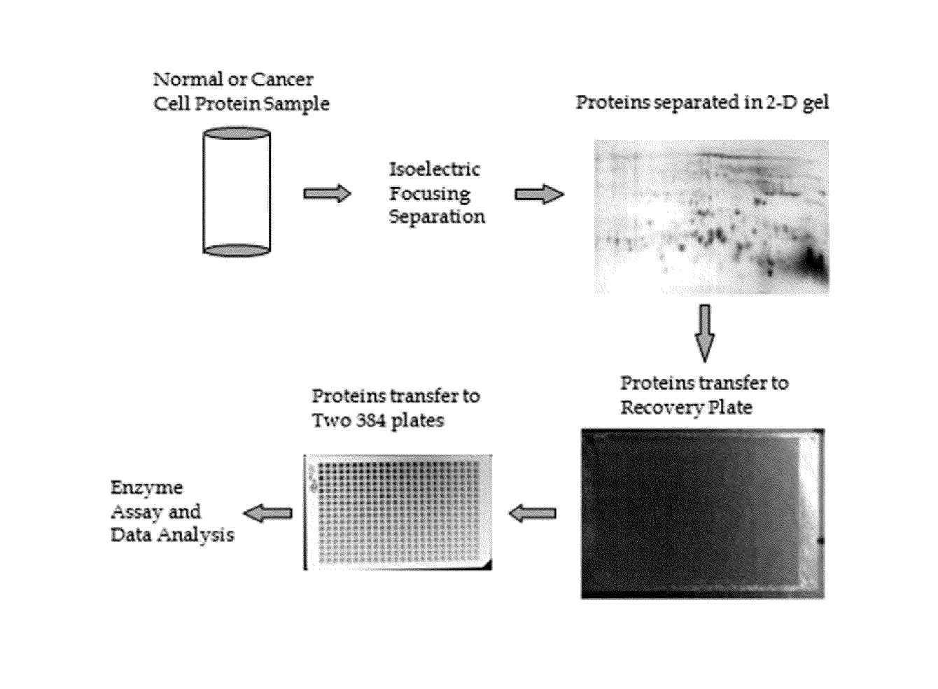

After the separation in the second dimension, the proteins were transferred into the Protein Elution Plate and samples from each well were used to run a standard SDS-PAGE gel to check for protein purity, as demonstrated in FIG. 2. Most of the proteins were transferred as shown by only a small amount detected in the gel after the transfer, where the remains represent the most abundant seed proteins, which would not be found in a typical cancer cell.

The transfer procedure is as follows: 1, after gel electrophoresis, the gel is placed on top of the Protein Elution Plate (PEP), which is filled with elution buffer. The bottom of the plate is attached with a material that is conductive. Either fixing aluminum foil or a dialysis membrane with adhesives accomplishes this. The protein elution is completed in a gel electrophoresis transfer tank with transfer current less than 400 mA and transfer for less than 12 hrs. After the transfer is complete, the assembled sandwich is frozen at −80° C. to prevent proteins spilling from one well to another. The plate is frozen, the gel is lifted and the PEP lyophilized. Following lyophilization, the wells of the PEP are filled with enzyme assay buffer and readied for analysis.

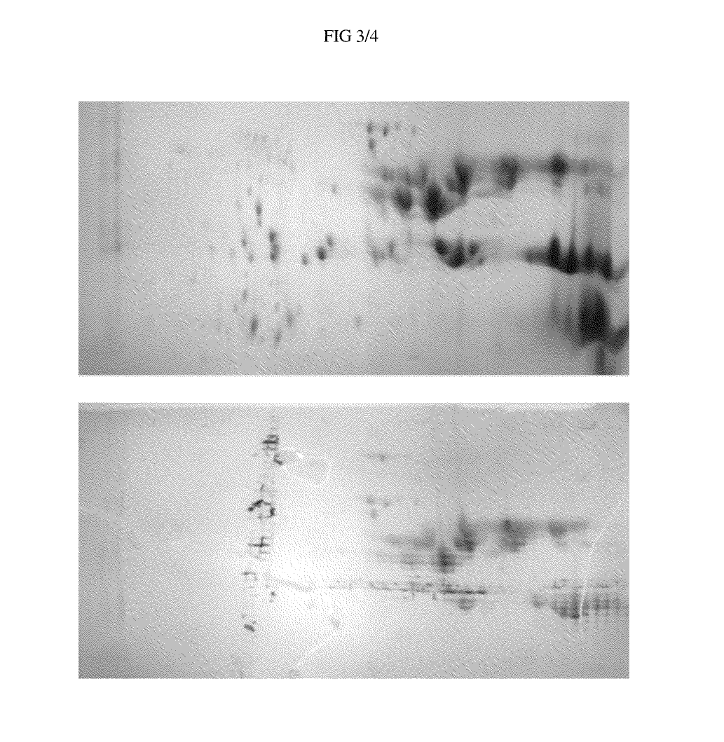

FIG. 3. shows the transfer of the separated proteins from 2-D Gel to PEP Recovery Plate. As indicated in FIG. 3, after protein transfer, the bulk of the proteins in the gel have been transferred to the PEP plate as reflected by the staining of the post-transfer gel and the detection of the proteins from the PEP wells.

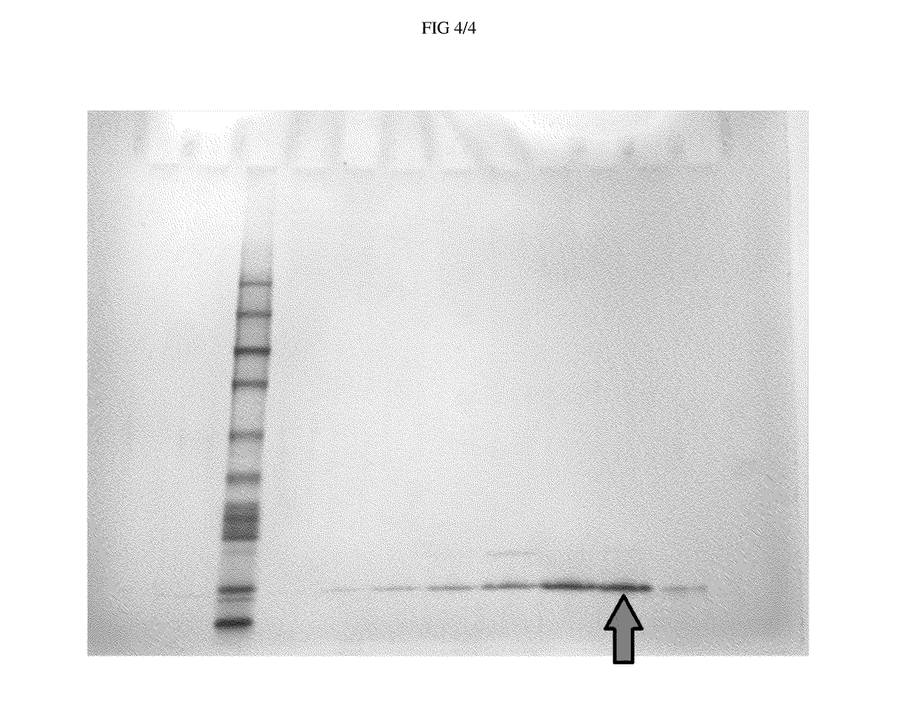

FIG. 4. indicates that protein recovered from individual wells of the PEP is relatively pure, suggesting that protein mixtures could be purified using this process.

Systematic Activity Analysis from Purified Protein Mixtures

Tables 5-8 illustrate the results from application of the claimed methods for different mixtures of proteins. The protein mixtures were obtained from both healthy and diseased cells, and subjected to the disclosed methods.

Table 5 shows the results from separating the proteins obtained from normal lung epithelial cells and analyzing them for NAD(+) reductase activity. Enzyme assays were performed on each well of the PEP plate and the results may be compiled to create a three-dimensional enzyme landscape.

Table 6 shows the results from separating the proteins obtained from stage-4 lung cancer cells and analyzing them for NAD(+) reductase activity. Enzyme assays were performed and the results may be compiled to create a three-dimensional enzyme landscape for the cancer cells.

Table 7 shows the results from separating the proteins obtained from normal lung epithelial cells and analyzing them for protein kinase activity. Enzyme assays were performed on each well of the PEP plate and the results may be compiled to create a three-dimensional enzyme landscape for the healthy cells.

Table 8 shows the results from separating the proteins obtained from stage-4 lung cancer cells and analyzing them for protein kinase activity. Enzyme assays were performed and the results may be compiled to create a three-dimensional enzyme landscape for the cancer cells.

TABLE 5

| | A | B | C | D | E | F | G | H | I | J | K | L |
|---|---|---|---|---|---|---|---|---|---|---|---|---|
| 1 | 0.243873 | 0.244341 | 0.248198 | 0.249344 | 0.23404 | 0.242617 | 0.250259 | 0.243655 | 0.249484 | 0.247129 | 0.240138 | 0.240894 |
| 2 | 0.242005 | 0.247381 | 0.248287 | 0.243155 | 0.248709 | 0.385189 | 0.266481 | 0.258323 | 0.254685 | 0.24614 | 0.241515 | 0.240853 |
| 3 | 0.251585 | 0.237126 | 0.248387 | 0.250041 | 0.248351 | 0.24739 | 0.249974 | 0.252521 | 0.250326 | 0.25074 | 0.251386 | 0.248059 |
| 4 | 0.239138 | 0.238651 | 0.246359 | 0.246356 | 0.247595 | 0.247218 | 0.245121 | 0.247262 | 0.248901 | 0.24349 | 0.249442 | 0.246672 |
| 5 | 0.297429 | 0.361982 | 0.287651 | 0.363334 | 0.294664 | 0.275046 | 0.265794 | 0.264384 | 0.252906 | 0.25149 | 0.247204 | 0.249818 |
| 6 | 0.241258 | 0.214229 | 0.249183 | 0.248487 | 0.256061 | 0.252134 | 0.264442 | 0.250058 | 0.256704 | 0.259813 | 0.247925 | 0.250477 |
| 7 | 0.255906 | 0.255708 | 0.265742 | 0.297361 | 0.262192 | 0.257172 | 0.294266 | 0.245958 | 0.239016 | 0.252861 | 0.247551 | 0.25081 |
| 8 | 0.242321 | 0.244572 | 0.247642 | 0.247356 | 0.244288 | 0.247198 | 0.241529 | 0.247331 | 0.250491 | 0.248529 | 0.247068 | 0.24691 |
| 9 | 0.244966 | 0.247595 | 0.250477 | 0.23947 | 0.245599 | 0.247817 | 0.238241 | 0.248654 | 0.253047 | 0.258439 | 0.252777 | 0.243823 |
| 10 | 0.27854 | 0.259032 | 0.284243 | 0.254296 | 0.261934 | 0.248523 | 0.247789 | 0.247209 | 0.249603 | 0.249556 | 0.24827 | 0.229723 |
| 11 | 0.24676 | 0.241551 | 0.244933 | 0.248584 | 0.244715 | 0.321969 | 0.248501 | 0.252499 | 0.257947 | 0.278007 | 0.234293 | 0.30034 |
| 12 | 0.253392 | 0.240228 | 0.246907 | 0.255917 | 0.247806 | 0.244341 | 0.248584 | 0.247173 | 0.245044 | 0.242414 | 0.238803 | 0.247038 |
| 13 | 0.255968 | 0.25713 | 0.291499 | 0.24409 | 0.251392 | 0.249394 | 0.339218 | 0.251224 | 0.246434 | 0.245488 | 0.246583 | 0.248684 |
| 14 | 0.252662 | 0.246121 | 0.251408 | 0.245151 | 0.250544 | 0.255581 | 0.24777 | 0.256906 | 0.26441 | 0.459879 | 0.324646 | 0.271156 |
| 15 | 0.257175 | 0.268064 | 0.266571 | 0.272344 | 0.25711 | 0.263664 | 0.241819 | 0.2592 | 0.25536 | 0.256696 | 0.255439 | 0.261034 |
| 16 | 0.261529 | 0.251557 | 0.265074 | 0.2559 | 0.26037 | 0.250181 | 0.261951 | 0.250407 | 0.25906 | 0.247889 | 0.267383 | 0.249341 |
| 17 | 0.233494 | 0.228881 | 0.236037 | 0.235326 | 0.235851 | 0.232923 | 0.250445 | 0.240664 | 0.247312 | 0.269254 | 0.266902 | 0.241974 |
| 18 | 0.238012 | 0.23736 | 0.237482 | 0.252269 | 0.238658 | 0.231726 | 0.23214 | 0.243601 | 0.238503 | 0.238251 | 0.233888 | 0.234804 |
| 19 | 0.244227 | 0.235334 | 0.232043 | 0.24409 | 0.240989 | 0.232201 | 0.239059 | 0.346757 | 0.234885 | 0.215047 | 0.380105 | 0.234038 |
| 20 | 0.236469 | 0.233832 | 0.2413 | 0.263043 | 0.223569 | 0.265486 | 0.265818 | 0.248163 | 0.38041 | 0.23493 | 0.260315 | 0.233689 |
| 21 | 0.235417 | 0.23833 | 0.295737 | 0.2983 | 0.223184 | 0.248579 | 0.220567 | 0.241564 | 0.124035 | 0.276391 | 0.235862 | 0.243116 |
| 22 | 0.237712 | 0.231851 | 0.239344 | 0.236641 | 0.231445 | 0.228343 | 0.294455 | 0.253497 | 0.247274 | 0.254964 | 0.238403 | 0.223865 |
| 23 | 0.241655 | 0.245912 | 0.652689 | 0.247769 | 0.255686 | 0.241742 | 0.237937 | 0.239578 | 0.313465 | 0.250875 | 0.266061 | 0.249132 |
| 24 | 0.233695 | 0.233966 | 0.241155 | 0.237579 | 0.240103 | 0.230563 | 0.233231 | 0.239073 | 0.237663 | 0.226031 | 0.250401 | 0.23885 |
| 25 | 0.244752 | 0.233376 | 0.241706 | 0.25005 | 0.24198 | 0.230233 | 0.229181 | 0.368784 | 0.235646 | 0.242968 | 0.234277 | 0.235339 |
| 26 | 0.233569 | 0.235776 | 0.241991 | 0.239581 | 0.230667 | 0.245011 | 0.235022 | 0.234266 | 0.228828 | 0.239826 | 0.237926 | 0.227134 |
| 27 | 0.229911 | 0.227137 | 0.247819 | 0.247393 | 0.231035 | 0.308871 | 0.24393 | 0.226664 | 0.279863 | 0.244873 | 0.240964 | 0.233025 |
| 28 | 0.215594 | 0.221609 | 0.240569 | 0.236417 | 0.235538 | 0.284135 | 0.243214 | 0.237493 | 0.243486 | 0.242406 | 0.250456 | 0.251922 |
| 29 | 0.239255 | 0.232899 | 0.251693 | 0.240798 | 0.248035 | 0.242885 | 0.250055 | 0.245493 | 0.245677 | 0.232059 | 0.254781 | 0.24961 |
| 30 | 0.250281 | 0.24311 | 0.514886 | 0.268422 | 0.2584 | 0.25105 | 0.240708 | 0.250738 | 0.239866 | 0.245493 | 0.232185 | 0.235862 |
| 31 | 0.231296 | 0.23223 | 0.252625 | 0.250434 | 0.255305 | 0.253985 | 0.256629 | 0.323948 | 0.262605 | 0.452309 | 0.274136 | 0.272069 |
| 32 | 0.21399 | 0.242822 | 0.229951 | 0.250975 | 0.255841 | 0.246008 | 0.251081 | 0.255878 | 0.247972 | 0.248199 | 0.245146 | 0.254112 |
| | M | N | O | P | Q | R | S | T | U | V | W | X |
| 1 | 0.237986 | 0.242971 | 0.25058 | 0.252948 | 0.246243 | 0.246276 | 0.246221 | 0.240832 | 0.235998 | 0.234522 | 0.239141 | 0.244164 |
| 2 | 0.228744 | 0.240537 | 0.245844 | 0.242041 | 0.238632 | 0.242074 | 0.239895 | 0.245402 | 0.229659 | 0.223637 | 0.231497 | 0.230845 |
| 3 | 0.241614 | 0.240007 | 0.246608 | 0.24782 | 0.248412 | 0.246248 | 0.252123 | 0.619362 | 0.27471 | 0.238246 | 0.240255 | 0.235619 |
| 4 | 0.247756 | 0.242227 | 0.24988 | 0.258496 | 0.234651 | 0.23839 | 0.245325 | 0.247892 | 0.233863 | 0.233395 | 0.245044 | 0.296779 |
| 5 | 0.239342 | 0.231634 | 0.44143 | 0.366699 | 0.317063 | 0.262505 | 0.249857 | 0.260585 | 0.25435 | 0.238385 | 0.241072 | 0.247898 |
| 6 | 0.247973 | 0.288879 | 0.336951 | 0.252721 | 0.250058 | 0.259736 | 0.264234 | 0.247823 | 0.244162 | 0.239868 | 0.257186 | 0.25426 |
| 7 | 0.254787 | 0.236198 | 0.244517 | 0.24471 | 0.24313 | 0.244374 | 0.246367 | 0.248378 | 0.232094 | 0.236999 | 0.239557 | 0.240007 |
| 8 | 0.244247 | 0.239563 | 0.246777 | 0.247556 | 0.257587 | 0.250835 | 0.254869 | 0.236717 | 0.244401 | 0.249843 | 0.233239 | 0.237741 |
| 9 | 0.245331 | 0.279468 | 0.249983 | 0.250673 | 0.245187 | 0.245687 | 0.251831 | 0.241414 | 0.227059 | 0.233739 | 0.232598 | 0.244062 |
| 10 | 0.238499 | 0.255171 | 0.240266 | 0.26552 | 0.247645 | 0.232799 | 0.278573 | 0.244432 | 0.223217 | 0.239972 | 0.240807 | 0.239882 |
| 11 | 0.26662 | 0.235107 | 0.254795 | 0.246312 | 0.244181 | 0.235811 | 0.248473 | 0.245394 | 0.23666 | 0.231024 | 0.25301 | 0.246016 |
| 12 | 0.232317 | 0.235525 | 0.249258 | 0.248239 | 0.233502 | 0.245842 | 0.239522 | 0.293873 | 0.264878 | 0.375909 | 0.261609 | 0.223692 |
| 13 | 0.240051 | 0.247576 | 0.249124 | 0.255241 | 0.242537 | 0.253361 | 0.25 | 0.25088 | 0.238964 | 0.235484 | 0.241951 | 0.243391 |
| 14 | 0.251719 | 0.248073 | 0.258027 | 0.24724 | 0.248932 | 0.249537 | 0.249947 | 0.254519 | 0.239454 | 0.257189 | 0.24516 | 0.232928 |
| 15 | 0.238572 | 0.257942 | 0.254065 | 0.252822 | 0.252434 | 0.274411 | 0.263598 | 0.267633 | 0.27994 | 0.271372 | 0.257104 | 0.265294 |
| 16 | 0.255086 | 0.248364 | 0.267822 | 0.259776 | 0.257891 | 0.249525 | 0.263229 | 0.250611 | 0.243765 | 0.244748 | 0.26061 | 0.259348 |
| 17 | 0.233711 | 0.238267 | 0.242327 | 0.238943 | 0.237195 | 0.233416 | 0.23566 | 0.241204 | 0.224805 | 0.220746 | 0.235975 | 0.227999 |
| 18 | 0.239089 | 0.231728 | 0.230659 | 0.236185 | 0.231107 | 0.312705 | 0.214446 | 0.252305 | 0.232503 | 0.221609 | 0.227139 | 0.226925 |
| 19 | 0.346503 | 0.232642 | 0.431113 | 0.220923 | 0.230883 | 0.229749 | 0.290702 | 0.240152 | 0.291112 | 0.20163 | 0.222361 | 0.219403 |
| 20 | 0.227263 | 0.231856 | 0.234812 | 0.278523 | 0.220364 | 0.230755 | 0.225802 | 0.231659 | 0.219961 | 0.217735 | 0.227491 | 0.227977 |
| 21 | 0.227951 | 0.235014 | 0.233518 | 0.244139 | 0.231501 | 0.242242 | 0.226437 | 0.242045 | 0.218931 | 0.217063 | 0.220551 | 0.228955 |
| 22 | 0.230143 | 0.24314 | 0.242968 | 0.232717 | 0.229141 | 0.228947 | 0.273756 | 0.242924 | 0.227956 | 0.212536 | 0.22541 | 0.213805 |
| 23 | 0.231862 | 0.233657 | 0.241136 | 0.239603 | 0.244004 | 0.23834 | 0.2404 | 0.236903 | 0.21744 | 0.231323 | 0.228134 | 0.343551 |
| 24 | 0.232738 | 0.23136 | 0.239067 | 0.235353 | 0.23591 | 0.234068 | 0.231256 | 0.230773 | 0.547706 | 0.216264 | 0.220673 | 0.225765 |
| 25 | 0.216289 | 0.230582 | 0.244323 | 0.244395 | 0.23203 | 0.423694 | 0.22963 | 0.27595 | 0.242346 | 0.300668 | 0.304683 | 0.225105 |
| 26 | 0.234925 | 0.209974 | 0.2383 | 0.245005 | 0.241409 | 0.256022 | 0.256157 | 0.240686 | 0.231253 | 0.230435 | 0.229624 | 0.222253 |
| 27 | 0.23195 | 0.232506 | 0.235514 | 0.234218 | 0.233196 | 0.241608 | 0.235337 | 0.231512 | 0.217461 | 0.211758 | 0.225533 | 0.226854 |
| 28 | 0.24412 | 0.240501 | 0.247061 | 0.263325 | 0.250038 | 0.354699 | 0.275853 | 0.269571 | 0.225805 | 0.227488 | 0.234256 | 0.248313 |
| 29 | 0.238924 | 0.239239 | 0.23878 | 0.244425 | 0.239059 | 0.240512 | 0.24071 | 0.247731 | 0.237436 | 0.252101 | 0.246375 | 0.242002 |
| 30 | 0.228791 | 0.230534 | 0.237541 | 0.236585 | 0.23722 | 0.239978 | 0.237644 | 0.225902 | 0.297663 | 0.236075 | 0.24437 | 0.230044 |
| 31 | 0.51545 | 0.300772 | 0.247755 | 0.258099 | 0.254781 | 0.260423 | 0.252342 | 0.252967 | 0.321653 | 0.241079 | 0.272239 | 0.248365 |
| 32 | 0.239502 | 0.247111 | 0.299291 | 0.283427 | 0.243626 | 0.266309 | 0.275395 | 0.355394 | 0.277698 | 0.242836 | 0.371483 | 0.267844 |

TABLE 6

| | A | B | C | D | E | F | G | H | I | J | K | L |
|---|---|---|---|---|---|---|---|---|---|---|---|---|
| 1 | 0.245344 | 0.243282 | 0.255023 | 0.254196 | 0.249836 | 0.249760 | 0.254583 | 0.257551 | 0.263071 | 0.261293 | 0.252451 | 0.253433 |
| 2 | 0.239817 | 0.250014 | 0.311576 | 0.466829 | 0.298905 | 0.253861 | 0.257593 | 0.248673 | 0.264281 | 0.245079 | 0.252906 | 0.244064 |
| 3 | 0.254250 | 0.242494 | 0.251183 | 0.253835 | 0.255309 | 0.249596 | 0.250210 | 0.260731 | 0.272333 | 0.255696 | 0.251332 | 0.258262 |
| 4 | 0.244872 | 0.244296 | 0.257451 | 0.253844 | 0.252957 | 0.247114 | 0.252904 | 0.267782 | 0.264550 | 0.252811 | 0.259328 | 0.249905 |
| 5 | 0.387327 | 0.281357 | 0.252707 | 0.246626 | 0.249135 | 0.244505 | 0.250400 | 0.251396 | 0.255934 | 0.249272 | 0.250965 | 0.249760 |
| 6 | 0.268239 | 0.253379 | 0.263840 | 0.278487 | 0.236599 | 0.275358 | 0.243354 | 0.254930 | 0.317807 | 0.255563 | 0.247455 | 0.244389 |
| 7 | 0.250065 | 0.247017 | 0.265339 | 0.255049 | 0.248303 | 0.260039 | 0.267140 | 0.253689 | 0.286815 | 0.294904 | 0.310259 | 0.295298 |
| 8 | 0.244947 | 0.245784 | 0.250132 | 0.247033 | 0.245275 | 0.243395 | 0.251632 | 0.249158 | 0.251935 | 0.264100 | 0.247491 | 0.261448 |
| 9 | 0.258177 | 0.236897 | 0.298384 | 0.273614 | 0.272513 | 0.255334 | 0.255512 | 0.257429 | 0.264229 | 0.259288 | 0.277751 | 0.253824 |
| 10 | 0.249593 | 0.249241 | 0.247061 | 0.237187 | 0.230877 | 0.235768 | 0.248712 | 0.263676 | 0.266785 | 0.242282 | 0.250797 | 0.294752 |
| 11 | 0.246465 | 0.240869 | 0.250012 | 0.247172 | 0.255280 | 0.247355 | 0.247972 | 0.246745 | 0.255475 | 0.257184 | 0.250705 | 0.242395 |
| 12 | 0.243387 | 0.245693 | 0.266025 | 0.250020 | 0.242014 | 0.244729 | 0.270962 | 0.252103 | 0.257824 | 0.249813 | 0.242461 | 0.246169 |
| 13 | 0.239858 | 0.238194 | 0.268251 | 0.272595 | 0.254227 | 0.343593 | 0.269172 | 0.372605 | 0.265157 | 0.286974 | 0.238804 | 0.293398 |
| 14 | 0.260036 | 0.261465 | 0.262743 | 0.275402 | 0.259833 | 0.264697 | 0.275699 | 0.477676 | 0.271834 | 0.316415 | 0.369016 | 0.286706 |
| 15 | 0.251419 | 0.264905 | 0.261448 | 0.272854 | 0.291050 | 0.261046 | 0.280151 | 0.265186 | 0.264463 | 0.437754 | 0.275651 | 0.275447 |
| 16 | 0.258276 | 0.252482 | 0.253483 | 0.329699 | 0.318994 | 0.251525 | 0.260233 | 0.255934 | 0.265018 | 0.252361 | 0.253545 | 0.252454 |
| 17 | 0.232984 | 0.239433 | 0.238671 | 0.236142 | 0.230019 | 0.308236 | 0.327886 | 0.387913 | 0.292262 | 0.251921 | 0.246873 | 0.238551 |
| 18 | 0.295181 | 0.324508 | 0.348265 | 0.277791 | 0.242644 | 0.248613 | 0.246950 | 0.276577 | 0.269023 | 0.251523 | 0.260045 | 0.243047 |
| 19 | 0.240179 | 0.240649 | 0.252479 | 0.242266 | 0.239918 | 0.237664 | 0.246219 | 0.248735 | 0.248691 | 0.251207 | 0.280249 | 0.261320 |
| 20 | 0.237062 | 0.242101 | 0.282709 | 0.248682 | 0.341707 | 0.243435 | 0.241764 | 0.238187 | 0.239525 | 0.242603 | 0.241283 | 0.249325 |
| 21 | 0.272450 | 0.262109 | 0.240182 | 0.251199 | 0.246031 | 0.239945 | 0.240854 | 0.237062 | 0.242847 | 0.242288 | 0.251787 | 0.250570 |
| 22 | 0.241608 | 0.240174 | 0.240155 | 0.237694 | 0.245407 | 0.244238 | 0.241214 | 0.242074 | 0.252168 | 0.252897 | 0.246419 | 0.286547 |
| 23 | 0.245548 | 0.242200 | 0.256981 | 0.520891 | 0.280519 | 0.270857 | 0.241937 | 0.253060 | 0.252625 | 0.243820 | 0.255690 | 0.257895 |
| 24 | 0.242724 | 0.241532 | 0.261317 | 0.305019 | 0.252513 | 0.243789 | 0.250408 | 0.253282 | 0.247918 | 0.253839 | 0.256825 | 0.243957 |
| 25 | 0.240840 | 0.242929 | 0.245371 | 0.243231 | 0.242740 | 0.245713 | 0.262071 | 0.258401 | 0.253437 | 0.266955 | 0.263215 | 0.255128 |
| 26 | 0.320525 | 0.542258 | 0.396776 | 0.264975 | 0.245766 | 0.240884 | 0.253024 | 0.262252 | 0.248299 | 0.255080 | 0.270310 | 0.264286 |
| 27 | 0.254386 | 0.243943 | 0.241759 | 0.240510 | 0.238299 | 0.236886 | 0.244748 | 0.239444 | 0.286835 | 0.250028 | 0.247632 | 0.256295 |
| 28 | 0.246308 | 0.240128 | 0.247693 | 0.235896 | 0.241948 | 0.239929 | 0.249509 | 0.249635 | 0.246181 | 0.245716 | 0.243977 | 0.238875 |
| 29 | 0.259631 | 0.250218 | 0.238049 | 0.250184 | 0.241050 | 0.246300 | 0.243910 | 0.242178 | 0.244266 | 0.257347 | 0.248138 | 0.251274 |
| 30 | 0.421236 | 0.258850 | 0.251859 | 0.247638 | 0.242946 | 0.246363 | 0.264525 | 0.237390 | 0.253642 | 0.229364 | 0.232710 | 0.384347 |
| 31 | 0.253865 | 0.251425 | 0.270211 | 0.262040 | 0.249233 | 0.247025 | 0.245504 | 0.240805 | 0.256338 | 0.244841 | 0.263900 | 0.251255 |
| 32 | 0.237086 | 0.233819 | 0.235041 | 0.242323 | 0.243476 | 0.242949 | 0.244739 | 0.240526 | 0.258634 | 0.244825 | 0.419679 | 0.257037 |
| | M | N | O | P | Q | R | S | T | U | V | W | X |
| 1 | 0.247033 | 0.348678 | 0.246321 | 0.246102 | 0.279454 | 0.257639 | 0.250730 | 0.258826 | 0.235001 | 0.236055 | 0.242167 | 0.237827 |
| 2 | 0.240872 | 0.244411 | 0.245159 | 0.246044 | 0.325189 | 0.242458 | 0.241682 | 0.242346 | 0.244955 | 0.237423 | 0.241718 | 0.244323 |
| 3 | 0.250741 | 0.258863 | 0.526050 | 0.302764 | 0.258763 | 0.261565 | 0.250218 | 0.249269 | 0.235320 | 0.242766 | 0.241520 | 0.251270 |
| 4 | 0.250355 | 0.241526 | 0.254083 | 0.247164 | 0.250215 | 0.242944 | 0.243194 | 0.241331 | 0.236244 | 0.230382 | 0.268207 | 0.241118 |
| 5 | 0.245983 | 0.243753 | 0.267297 | 0.268534 | 0.255869 | 0.247944 | 0.253376 | 0.247089 | 0.243310 | 0.235966 | 0.251713 | 0.239705 |
| 6 | 0.247599 | 0.242389 | 0.246545 | 0.246393 | 0.246426 | 0.251326 | 0.247291 | 0.266753 | 0.248500 | 0.237342 | 0.242870 | 0.241685 |
| 7 | 0.247494 | 0.251323 | 0.252196 | 0.262737 | 0.247272 | 0.253706 | 0.234521 | 0.257329 | 0.235906 | 0.235922 | 0.238009 | 0.245762 |
| 8 | 0.244464 | 0.246698 | 0.251360 | 0.240915 | 0.263033 | 0.234144 | 0.321364 | 0.282276 | 0.262723 | 0.247424 | 0.249261 | 0.391578 |
| 9 | 0.256852 | 0.260763 | 0.257872 | 0.258815 | 0.249788 | 0.256520 | 0.284257 | 0.270664 | 0.244422 | 0.301404 | 0.253565 | 0.264787 |
| 10 | 0.229908 | 0.299913 | 0.282722 | 0.252204 | 0.251525 | 0.254360 | 0.252100 | 0.252625 | 0.240855 | 0.239695 | 0.244001 | 0.253582 |
| 11 | 0.245358 | 0.229087 | 0.236583 | 0.266504 | 0.256736 | 0.254859 | 0.266994 | 0.244894 | 0.385854 | 0.245480 | 0.252184 | 0.257309 |
| 12 | 0.240156 | 0.245460 | 0.246822 | 0.257295 | 0.248581 | 0.246501 | 0.296424 | 0.253632 | 0.276312 | 0.264281 | 0.280445 | 0.235577 |
| 13 | 0.254636 | 0.262625 | 0.263333 | 0.274627 | 0.250179 | 0.251881 | 0.264694 | 0.260594 | 0.262165 | 0.258980 | 0.251775 | 0.263909 |
| 14 | 0.290084 | 0.261947 | 0.296431 | 0.457029 | 0.246473 | 0.311264 | 0.258256 | 0.287765 | 0.248871 | 0.257298 | 0.355632 | 0.247436 |
| 15 | 0.315047 | 0.261660 | 0.262349 | 0.264388 | 0.277870 | 0.295108 | 0.285865 | 0.279717 | 0.272545 | 0.288259 | 0.296962 | 0.314940 |
| 16 | 0.272275 | 0.238409 | 0.253725 | 0.257938 | 0.253371 | 0.257048 | 0.262680 | 0.766156 | 0.720299 | 0.918827 | 0.519300 | 1.008008 |
| 17 | 0.226344 | 0.229271 | 0.241029 | 0.240663 | 0.243105 | 0.238774 | 0.237333 | 0.256272 | 0.244538 | 0.241540 | 0.295878 | 0.322825 |
| 18 | 0.236507 | 0.231481 | 0.254053 | 0.351249 | 0.243501 | 0.516896 | 0.276627 | 0.239678 | 0.224546 | 0.223245 | 0.229162 | 0.223886 |
| 19 | 0.264629 | 0.286598 | 0.254614 | 0.247541 | 0.240111 | 0.240788 | 0.238679 | 0.238698 | 0.228280 | 0.224320 | 0.230453 | 0.231246 |
| 20 | 0.265715 | 0.258014 | 0.242109 | 0.237995 | 0.234931 | 0.235853 | 0.232410 | 0.229499 | 0.221993 | 0.231473 | 0.232164 | 0.234500 |
| 21 | 0.240463 | 0.258003 | 0.265843 | 0.366605 | 0.267783 | 0.237344 | 0.236781 | 0.233518 | 0.224914 | 0.221766 | 0.235222 | 0.224572 |
| 22 | 0.258014 | 0.263796 | 0.294491 | 0.263131 | 0.233086 | 0.264009 | 0.239504 | 0.237623 | 0.225236 | 0.233161 | 0.247028 | 0.230051 |
| 23 | 0.262803 | 0.253741 | 0.296034 | 0.285531 | 0.250449 | 0.236621 | 0.239014 | 0.249986 | 0.248318 | 0.236981 | 0.246134 | 0.229688 |
| 24 | 0.240381 | 0.231505 | 0.256675 | 0.246277 | 0.251913 | 0.238364 | 0.239054 | 0.233019 | 0.238285 | 0.230360 | 0.232847 | 0.230432 |
| 25 | 0.231059 | 0.243954 | 0.248738 | 0.247727 | 0.238897 | 0.225085 | 0.236518 | 0.240327 | 0.229220 | 0.234675 | 0.231428 | 0.253145 |
| 26 | 0.247491 | 0.244748 | 0.248891 | 0.245236 | 0.247957 | 0.245371 | 0.270813 | 0.245865 | 0.231388 | 0.253243 | 0.229558 | 0.228609 |
| 27 | 0.248702 | 0.404118 | 0.303988 | 0.246457 | 0.233760 | 0.234553 | 0.235589 | 0.238296 | 0.230480 | 0.227450 | 0.232662 | 0.230832 |
| 28 | 0.238052 | 0.231161 | 0.241975 | 0.235173 | 0.238350 | 0.234182 | 0.241502 | 0.236875 | 0.225929 | 0.225882 | 0.580565 | 0.251059 |
| 29 | 0.235095 | 0.237661 | 0.243660 | 0.283977 | 0.309873 | 0.272653 | 0.467965 | 0.255820 | 0.227294 | 0.476374 | 0.238223 | 0.248755 |
| 30 | 0.242691 | 0.238660 | 0.245363 | 0.245054 | 0.242005 | 0.240796 | 0.240321 | 0.236889 | 0.230053 | 0.233599 | 0.234287 | 0.236258 |
| 31 | 0.234572 | 0.237284 | 0.242556 | 0.238845 | 0.232209 | 0.236418 | 0.248635 | 0.244574 | 0.463978 | 0.263888 | 0.251548 | 0.241792 |
| 32 | 0.284814 | 0.249755 | 0.244048 | 0.241204 | 0.281145 | 0.256567 | 0.244910 | 0.321189 | 0.235424 | 0.237618 | 0.246662 | 0.239073 |

TABLE 7

| | A | B | C | D | E | F | G | H |
|---|---|---|---|---|---|---|---|---|
| 1 | 194939 | 869743 | 362513 | 457093 | 63743 | 312873 | 214453 | 534243 |
| 2 | 192402 | 1926723 | 403853 | 543843 | −301157 | 326903 | 74273 | 695103 |
| 3 | 184755 | 703523 | 214413 | 440013 | −32827 | 343613 | 180233 | 543493 |
| 4 | 198659 | −548457 | 169493 | 353303 | 55233 | 288303 | 59993 | 478193 |
| 5 | 743413 | 49473 | 133753 | 369713 | 71913 | 361053 | 84573 | −1042417 |
| 6 | 492293 | −270547 | −85837 | 602503 | 78753 | 272063 | 145073 | −1616377 |
| 7 | 269273 | −201117 | −191857 | 853323 | 209123 | 830993 | 126073 | −760137 |
| 8 | 284083 | 425633 | −63177 | 854043 | 140883 | 772433 | −116697 | −976207 |
| 9 | 23573 | 818993 | 276993 | 421733 | 163383 | −77487 | 39573 | −2702447 |
| 10 | 119423 | 435543 | 249163 | 649023 | 120993 | 418583 | −120027 | −1598177 |
| 11 | −9197 | 332133 | 214233 | 433253 | 243633 | −689337 | 655203 | −186357 |
| 12 | 24973 | 250513 | 224453 | 149123 | 194813 | −480617 | 117983 | −90977 |
| 13 | −1003937 | 264183 | 357793 | 357823 | 164263 | −107367 | −76997 | −283137 |
| 14 | 1046003 | 330253 | 262723 | 311723 | 70883 | −604107 | −863087 | −647967 |
| 15 | 199633 | 394153 | 214433 | 570093 | 71363 | −831737 | −620597 | −1030637 |
| 16 | 292433 | 354353 | 230813 | 213393 | 538693 | −966137 | −437557 | −647837 |
| 17 | 426533 | 523863 | 269903 | −121107 | −299967 | −178867 | −20817 | −432627 |
| 18 | 404803 | 624293 | 128693 | −220157 | −410187 | −395067 | 122563 | 78953 |
| 19 | 220203 | 660143 | 183493 | −273107 | −617177 | −478587 | 109173 | 39543 |
| 20 | 346003 | 722393 | −230637 | −365967 | 14103 | −380537 | 308583 | −474287 |
| 21 | 306863 | 62083 | −245547 | −250867 | −25007 | −554617 | 14283 | −371417 |
| 22 | 609583 | 193963 | −637787 | −317117 | 34393 | −491117 | 193453 | −255277 |
| 23 | 281833 | 81923 | 483773 | −181067 | 137163 | −256297 | 367663 | −332737 |
| 24 | 463743 | 155153 | −281917 | −246267 | 140653 | 90823 | 306853 | −398657 |
| 25 | 32653 | 918237 | 287317 | −84143 | −57253 | 284047 | 394577 | 238927 |
| 26 | 25321 | 551927 | 363857 | −20093 | 107687 | 165527 | 126307 | 185777 |
| 27 | 12345 | 793397 | −678653 | 330597 | 110767 | 167937 | 214507 | 243137 |
| 28 | 23123 | 385657 | −303273 | 499567 | 226907 | 535757 | 312597 | −595203 |
| 29 | 287907 | 275927 | −358973 | 152737 | 139417 | 222197 | 373197 | −534563 |
| 30 | 312407 | 93157 | −371563 | −38643 | 262437 | 133417 | 56377 | −258063 |
| 31 | 252747 | −136133 | −97023 | 122167 | 177727 | 80317 | 181177 | −57383 |
| 32 | 298607 | −137763 | 109767 | 41697 | 127817 | −1044063 | 130097 | −11633 |
| 33 | 210127 | −474523 | 61317 | 82267 | 519747 | 512367 | −15373 | 11287 |
| 34 | 92357 | −52183 | 112547 | −61723 | 202187 | −5043 | −198043 | 84737 |
| 35 | −1247663 | −260043 | 54117 | −43143 | −2673 | −1720433 | 5277 | 1407 |
| 36 | −620083 | −429633 | 75697 | 86177 | 168977 | −620683 | 115187 | 170897 |
| 37 | −258913 | 142607 | 100367 | 349317 | 214027 | −339133 | 145287 | 156607 |
| 38 | 39287 | 198917 | 80157 | 169097 | 97197 | −38063 | 52977 | 97857 |
| 39 | 30847 | 227937 | 158097 | 206237 | −125583 | −299143 | 138017 | 47207 |
| 40 | 103977 | −10733 | 211767 | −20753 | −602843 | −571723 | −14473 | 23337 |
| 41 | 157447 | 104097 | −8533 | −1247283 | −491153 | −159213 | −3573 | 146947 |
| 42 | 20147 | 196057 | 72547 | −323453 | −120753 | −242913 | 103837 | 172697 |
| 43 | 29757 | 256547 | −71163 | −450463 | −60783 | −116523 | 83727 | −38313 |
| 44 | 42417 | 87917 | −112223 | −40823 | 16807 | −96473 | 208017 | −102433 |
| 45 | 22797 | 43137 | −184853 | −413053 | −96013 | −27233 | −135823 | 252777 |
| 46 | 144837 | −26923 | −221913 | −291253 | −163303 | −141523 | 668267 | 2897 |
| 47 | 156827 | −74883 | −261333 | −324773 | 53067 | 62917 | 275537 | −20283 |
| 48 | 227367 | 81257 | −1308433 | −1737473 | 34137 | 237527 | 295537 | 63587 |
| | I | J | K | L | M | N | O | P |
| 1 | 124963 | −1168977 | 901213 | 291243 | 16343 | 12183 | 345753 | 618053 |
| 2 | 199753 | −1017517 | 677733 | 377593 | −357317 | 158543 | 508853 | 424283 |
| 3 | −28287 | −1375397 | 811513 | 665403 | −590277 | 254303 | 459243 | 584683 |
| 4 | −537527 | −2946057 | 450323 | 290943 | −163427 | 182313 | 485953 | 622463 |
| 5 | −595417 | −1289247 | 400153 | 113363 | −717487 | 115353 | 495583 | 497703 |
| 6 | −73897 | −672597 | 375363 | 941173 | −549697 | 312483 | 425153 | 346373 |
| 7 | −1085557 | −1965037 | 146633 | 641933 | −355807 | 339043 | 205323 | 310833 |
| 8 | −241457 | 877683 | 80043 | −1246577 | 54423 | 386933 | 308153 | 244473 |
| 9 | −10707 | −2866377 | −357337 | −167637 | −19307 | 333333 | 312833 | 293583 |
| 10 | 57523 | −1668347 | 216643 | −910057 | 96063 | 150973 | 331513 | 45423 |
| 11 | 156583 | −997017 | −1152077 | −213517 | 531843 | 650373 | 112263 | −254937 |
| 12 | 335883 | −248227 | −670037 | 288303 | 306123 | 74983 | 439853 | −440527 |
| 13 | 173193 | −1537527 | 200493 | 30593 | 109433 | 411073 | 260843 | −473657 |
| 14 | 94413 | −1716787 | −1017537 | 128273 | 128553 | 55873 | 436503 | −266747 |
| 15 | 110463 | −3960467 | −440107 | −52467 | 66343 | 249683 | −232277 | −492337 |
| 16 | 129763 | −219837 | −201287 | 761643 | −10327 | 117843 | 150563 | 549853 |
| 17 | 618973 | −852117 | −39047 | 599513 | 254153 | −910407 | −234067 | 311243 |
| 18 | 217403 | −398577 | −126927 | 411213 | 277473 | −1127887 | −56507 | 349293 |
| 19 | 292683 | −349757 | −108597 | 272163 | 382713 | −755217 | 186763 | 499403 |
| 20 | 313273 | −219257 | −355387 | 177733 | 191403 | −1157977 | 293433 | 142853 |
| 21 | −272617 | 32893 | 221743 | 298093 | −821537 | −895637 | −151177 | 111933 |
| 22 | −277287 | −153457 | −49947 | 17003 | −1841427 | −432997 | 27513 | 255173 |
| 23 | −1461897 | 32533 | 111723 | 84093 | −817067 | 1863 | 5383 | 460253 |
| 24 | −976107 | 276223 | −224037 | 31803 | −260357 | 263043 | 263583 | 238873 |
| 25 | 274247 | 254697 | 194257 | 228917 | 4137 | 119117 | −200343 | 166017 |
| 26 | 126887 | 40617 | 38787 | 178997 | 133057 | 106727 | −984173 | 155557 |
| 27 | 113767 | 87667 | 254727 | 285797 | 109637 | 117707 | −694943 | 85447 |
| 28 | 93187 | 116727 | 222657 | 110357 | 156617 | 78117 | −577143 | 148817 |
| 29 | 146377 | 125457 | 275137 | 469727 | −67903 | 93707 | −41043 | 114507 |
| 30 | 40137 | 210927 | 288617 | 89997 | 62467 | −16863 | −163913 | 189277 |
| 31 | −10973 | 149667 | 235427 | 71697 | −1126053 | −2503 | 119007 | 151037 |
| 32 | 77187 | 171587 | 293307 | −107483 | −303183 | 70427 | −23203 | 166597 |
| 33 | 148967 | 134437 | 37067 | 340767 | −64123 | 98697 | 204297 | 77987 |
| 34 | 149927 | 308277 | 51407 | 484867 | −568513 | 33557 | −23093 | 122067 |
| 35 | 289817 | 192347 | −578283 | 122467 | −329723 | −100973 | 141467 | 131047 |
| 36 | 192847 | −5503 | −492103 | 610867 | 31717 | 382607 | 987 | 155937 |
| 37 | 869397 | 186447 | 255667 | 228567 | 7067 | 26277 | 157097 | −1223 |
| 38 | 394677 | 82287 | 705167 | −99033 | 42527 | 35787 | 105427 | 36877 |
| 39 | 466857 | −58563 | −1033133 | −69883 | −97053 | −228023 | 241107 | −58663 |
| 40 | 84427 | −176413 | −905033 | 12667 | −28973 | −208443 | −183843 | −342923 |
| 41 | 112487 | −108193 | −435103 | 4907 | −112623 | −161183 | −67053 | −304813 |
| 42 | −754203 | 22207 | −365363 | 51167 | 9027 | −200563 | −217703 | −453993 |
| 43 | −446203 | 55597 | −335263 | −47193 | 108617 | −35573 | −131733 | −574313 |
| 44 | −35913 | −28283 | −2013 | −36493 | 134467 | −11543 | −242103 | −1418723 |
| 45 | −1146533 | −96563 | −128373 | 270117 | 2717 | −39923 | −34523 | −969503 |
| 46 | −1042713 | −220993 | −71123 | −2703 | 101857 | −146133 | −128343 | −110713 |
| 47 | −667953 | −141263 | 126677 | −191433 | 5917 | −1685093 | −84583 | 25677 |
| 48 | −235133 | −88803 | 27137 | −54053 | −13783 | −242683 | −54103 | 64197 |

TABLE 8

| | A | B | C | D | E | F | G | H |
|---|---|---|---|---|---|---|---|---|
| 1 | 198764 | 267127 | 492207 | 201407 | 16537 | 283777 | −48213 | −1310653 |
| 2 | 184261 | 59147 | 237047 | 175807 | −231673 | 152137 | 39997 | −773763 |
| 3 | 161525 | −60553 | 502927 | 164817 | −301363 | 111747 | −651723 | −247383 |
| 4 | 629057 | −147163 | 301017 | 114197 | −531003 | 413477 | −327223 | 9007 |
| 5 | 588647 | −19213 | 411757 | 135697 | −371323 | 241587 | 81927 | 12957 |
| 6 | 398167 | −246203 | 268827 | 142767 | −1340763 | 106267 | −243643 | 171717 |
| 7 | 398457 | −1029883 | 120547 | 97077 | −536503 | −85253 | −120123 | 128167 |
| 8 | 329257 | −188693 | 96807 | 176897 | −152393 | 150097 | −541643 | 155457 |
| 9 | 653817 | 195237 | 12687 | 127257 | −72953 | −281313 | −621483 | 187937 |
| 10 | 445557 | 262107 | 128017 | 200507 | −199013 | −231143 | −1563523 | 100127 |
| 11 | 422757 | 201727 | 142907 | 123077 | 36407 | −344873 | −612803 | −739863 |
| 12 | 546837 | 305277 | 2847 | 165617 | 74327 | −65673 | 74327 | 210377 |
| 13 | 367307 | 223927 | 87827 | 74787 | 144447 | −343743 | −213593 | −836183 |
| 14 | 414547 | 260397 | −32083 | 37577 | 137607 | −3043 | −59983 | −227283 |
| 15 | 348287 | 192837 | −472553 | 21747 | −30293 | 33397 | −350143 | −164203 |
| 16 | 340847 | 204177 | −142373 | 49827 | 330017 | −268743 | 727567 | −1214453 |
| 17 | 386457 | 397357 | −148733 | 184067 | 230047 | −361553 | −421653 | −242493 |
| 18 | 206167 | 183617 | −1189023 | −24713 | 350197 | −650013 | −173053 | −138883 |
| 19 | 315397 | 164957 | −413463 | 8867 | −190533 | −3002073 | −1911593 | −769463 |
| 20 | 331887 | 94997 | 51877 | −64563 | −60703 | −443503 | 1755667 | −1639053 |
| 21 | 126447 | 155587 | −57063 | −244083 | −148943 | −50763 | −617983 | −1068993 |
| 22 | 35537 | 266337 | 59177 | −40533 | 99397 | 30907 | 17147 | −466583 |
| 23 | 278487 | 192247 | 429507 | 100817 | 205047 | 23477 | −355453 | −615393 |
| 24 | 126327 | 312537 | 170187 | −47813 | −5713 | 251007 | −96583 | −565883 |
| 25 | 80256 | 125475 | 41365 | 20495 | 305855 | 208395 | 4255 | −901675 |
| 26 | 115244 | −97415 | 93825 | 102835 | 548225 | 145055 | 45935 | 62785 |
| 27 | 95383 | −74585 | 20535 | 77545 | −10705 | 96365 | 96825 | −217185 |
| 28 | 26745 | −575815 | 107955 | 68685 | −364215 | 102815 | 129235 | 149695 |
| 29 | 138465 | −212525 | 40035 | 16645 | −679075 | 30275 | 10135 | −10745 |
| 30 | 55805 | −14465 | 28535 | −130425 | −443715 | 34365 | −62275 | −21985 |
| 31 | −48575 | −771195 | 145965 | 35455 | −340185 | 3165 | −60675 | 205 |
| 32 | −696975 | −529065 | −37195 | −118655 | −468635 | −250685 | −131065 | −49595 |
| 33 | 68185 | −542115 | 35945 | −8215 | −244385 | −43385 | 130625 | −15105 |
| 34 | 33705 | −200855 | −185025 | −34735 | −181805 | −139665 | 438555 | −81285 |
| 35 | 131035 | −238575 | −283505 | −56445 | −66365 | −601995 | 11535 | −78705 |
| 36 | −62025 | −162405 | −93535 | 131895 | −192255 | 6815 | 78075 | −8775 |
| 37 | 156225 | −81885 | −288325 | 18645 | −199405 | 543125 | 265715 | −18825 |
| 38 | 180185 | −20755 | −80065 | 339765 | −336155 | −1875005 | 183205 | 30515 |
| 39 | 124245 | −74875 | 554035 | 55895 | 3015 | −447485 | 45835 | −81115 |
| 40 | −13425 | −12885 | 107135 | −27715 | −136265 | −969295 | −65115 | −136985 |
| 41 | −48505 | −1615 | −97195 | 31305 | −54435 | −767135 | −361695 | −53815 |
| 42 | −139545 | −37005 | −449185 | −23645 | −133135 | −64545 | −70765 | 42755 |
| 43 | 128905 | −44925 | −285825 | −67865 | −78625 | −191205 | −187585 | 163605 |
| 44 | 56225 | −55365 | −41955 | −218255 | −118075 | −271335 | −80825 | −121415 |
| 45 | 22655 | −135055 | −189515 | −391135 | −178925 | −180685 | 2395 | −125145 |
| 46 | −37725 | −86585 | −152415 | −316075 | −180805 | −119725 | −99415 | −110265 |
| 47 | −46695 | −64205 | −52345 | 91235 | 107745 | −87725 | −254815 | 320465 |
| 48 | −157535 | 24375 | −71295 | 458205 | 21635 | −13555 | 643595 | 208765 |
| | I | J | K | L | M | N | O | P |
| 1 | −93463 | 248567 | 202567 | −17043 | 244857 | 519407 | 115017 | 257837 |
| 2 | −791333 | 165497 | 254337 | 66627 | 129247 | 313837 | 89207 | 359207 |
| 3 | −1151153 | 191837 | 283767 | −908273 | 131797 | 176797 | −264973 | 146997 |
| 4 | −1552073 | 15597 | −22043 | −988743 | 79757 | 178427 | −1108993 | 203027 |
| 5 | 16807 | 219597 | −81513 | 56107 | −44493 | 165727 | −499913 | 68687 |
| 6 | 33557 | −32423 | 44987 | 67097 | −177373 | 125697 | −648753 | −22533 |
| 7 | 125787 | 161457 | 127497 | −57123 | 79537 | −168433 | −55443 | 318617 |
| 8 | 163527 | 377477 | 122367 | 168647 | 144387 | 74537 | 98627 | 282367 |
| 9 | 125547 | 306357 | −53423 | 148677 | 237177 | 156877 | 166437 | 97727 |
| 10 | 106817 | −181693 | −369643 | 207987 | 465867 | 23437 | 198657 | 81867 |
| 11 | 75467 | 805517 | −1122353 | 75867 | 733767 | 188777 | 266967 | 88167 |
| 12 | −609573 | 745067 | −522333 | 209317 | −386313 | 234197 | 710447 | 150927 |
| 13 | −713743 | 816267 | 23547 | 199817 | −594703 | 141717 | 229217 | 177887 |
| 14 | −93283 | −253893 | 134667 | 295487 | −313483 | 234497 | 302257 | 828867 |
| 15 | −105543 | −834673 | 59777 | 92537 | −848903 | 13927 | 96067 | −732723 |
| 16 | −188223 | −535553 | −11903 | 85977 | −736913 | 256357 | 125257 | −422073 |
| 17 | −169273 | −158363 | 108457 | 208927 | −484153 | 586767 | 90377 | −900753 |
| 18 | −42853 | −88973 | −39353 | 29807 | −152853 | 108027 | −58343 | −550423 |
| 19 | 1809067 | −1332553 | 1834937 | −1207343 | −50793 | −20713 | 101897 | −215393 |
| 20 | −125313 | 108787 | −152653 | 4867 | 55187 | 94457 | 42327 | −37723 |
| 21 | −108483 | 233227 | 61597 | −88753 | 174337 | 11307 | 152277 | 144277 |
| 22 | 158687 | 227257 | 344507 | −48863 | 148717 | 280477 | 150547 | 183677 |
| 23 | 83187 | 294567 | 823147 | 212767 | 133817 | 200307 | 248597 | 277297 |
| 24 | −633 | 189727 | −275403 | 141387 | 488267 | 183607 | 347617 | 244397 |
| 25 | 166735 | 102535 | −202715 | 102225 | 179845 | −398915 | 362425 | 425305 |
| 26 | 171255 | 128125 | 671775 | 590355 | 103805 | −249755 | 1639575 | 287805 |
| 27 | −32425 | 155535 | 169835 | 249175 | 105685 | −7955 | −1026265 | 265255 |
| 28 | 58595 | −116515 | −5395 | −9265 | 71015 | 697755 | 85785 | 308595 |
| 29 | 384725 | 639445 | 2215 | 54875 | 210435 | 168075 | 66735 | 193695 |
| 30 | 2355 | 62565 | 2765 | 71505 | 133275 | 8495 | 59025 | 170455 |
| 31 | −54765 | 155185 | −44635 | −94065 | 92755 | −233485 | 141375 | 182605 |
| 32 | 1395 | −62215 | 8305 | −1515 | 52185 | 21475 | 165665 | 94645 |
| 33 | −1535 | −50475 | −24315 | −98675 | 45525 | 166155 | −16795 | 246445 |
| 34 | −84705 | 56095 | 74195 | 100195 | −215525 | −10595 | 56645 | 225065 |
| 35 | 632925 | 188745 | 355935 | 480685 | 170955 | 44795 | 80745 | 308695 |
| 36 | −129045 | 118825 | 74845 | 66465 | 454625 | 59155 | −6435 | 112535 |
| 37 | −520525 | 93055 | −127385 | −724765 | 42045 | −50145 | 669705 | −4795 |
| 38 | −228575 | 46385 | 188125 | −366005 | 179185 | 70715 | 270145 | 135895 |
| 39 | −583875 | −14515 | −65735 | −147045 | 162575 | 18845 | 66815 | 136625 |
| 40 | −304785 | 585205 | −84255 | −189655 | 26305 | 125135 | 11005 | 108635 |
| 41 | −368635 | −24705 | −76525 | −624735 | −153685 | 86145 | −175 | 137935 |
| 42 | −195435 | −39785 | 144355 | −166995 | −79495 | −14775 | −87505 | 38465 |
| 43 | −98155 | −78395 | 140995 | −380645 | 58555 | 116735 | 39435 | 259185 |
| 44 | −26945 | −56815 | 103915 | 87265 | 905 | −7735 | −41435 | 83885 |
| 45 | −111835 | −54335 | 825 | −143305 | −64095 | −27695 | −154035 | 95715 |
| 46 | −45685 | −95105 | 153575 | 1245 | −5005 | −55255 | 3085 | 131875 |
| 47 | 93645 | 631645 | −43005 | 28825 | 84085 | 228665 | 108745 | 41765 |
| 48 | −22255 | −187345 | 28125 | 37975 | 69705 | 238585 | 315495 | −215835 |

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinence of the cited references.

### REFERENCES

1. Rizk, N. I.; F. Valentich. Matrix recovery electrophoresis apparatus. U.S. Pat. No. 4,181,594. 1980.
2. Love, J. D.; Elliott, M. T.; Morgan, P. L. Process and apparatus for conducting electrophoresis and transfer. U.S. Pat. No. 4,726,889. 1988.
3. Andersen, P. Apparatus and process for electroelution of a gel containing charged macromolecules. U.S. Pat. No. 5,840,169. 1998.
4. Gulle, H.; B. Schoel.; S. H. E. Kaufmann. 1990. Direct blotting with viable cells of protein mixtures separated by two-dimensional gel electrophoresis. J. Immunological Methods, 133, 253-261.
5. Gulle, H.; S. H. E. Kaufmann.; K. M. Moriarty. 1993. Rapid electroelution of two-dimensionally separated protein mixtures: Its use in in vitro assays of T cell activities. Electrophoresis, 14, 902-908.
6. Jungblut, P.; B, Thiede.; U. Zimny-Arndt.; E-C. Muller.; C. Scheler.; B. Wittmann-Liebold. and A. Otto. 1996. Resolution power of two-dimensional electrophoresis and identification of proteins from gels. Electrophoresis, 17, 839-847.
7. Anderson, N. G. and N. L. Anderson. 1996. Twenty years of two-dimensional electrophoresis: Past, present and future. Electrophoresis, 17, 443-453.
8. Langen, H.; D. Rader.; J-F. Juranville. and M. Fountoulakis. 1997. Effect of protein application mode and acrylamide concentration on the resolution of protein spots separated by two-dimensional gel electrophoresis. Electrophoresis, 18, 2085-2090.
9. Gorg, A.; G. Boguth.; C. Obermaier.; A. Posch and W. Weiss. 1995. Two-dimensional polyacrylamide gel electrophoresis with immobilized pH gradients in the first dimension (IPG-Dalt): The state of the art and the controversy of vertical versus horizontal system. Electrophoresis, 16, 1079-1086.
10. Tsugita, A.; M. Kamo.; T. Kawakami. And Y. Ohki. 1996. Two-dimensional electrophoresis of plant proteins and standardization of gel patterns. Electrophoresis, 17, 855-865.
11. Nestler, H. P. and A. Doseff. 1997. A two-dimensional, diagonal sodium dodecylsulfate-polyacrylamide gel electrophoresis technique to screen for protease substrates in protein mixtures. Analytical Chemistry, 251, 122-125.
12. Naryzhny, S. N. 1997. “Active” two-dimensional electrophoresis of rat liver DNA-polymerase. Electrophoresis, 18, 553-556.
13. Kristensen, D. B.; M. Inamatsu. And K. Yoshizato. 1997. Elution concentration of proteins cut from two-dimensional polyacrylamide gels using Pasteur pipettes. Electrophoresis, 18, 2078-2084.

What is claimed is:

1. A method for purifying and characterizing proteins from a mixture comprising:
passing the mixture through at least two orthogonal separations under conditions that preserve protein activity;
eluting the purified proteins into individual wells of a protein elution plate; and
assaying the purified proteins in each well for protein activity.

2. The method according to claim 1, wherein the proteins in the mixture are purified in a first separation according to their isoelectric points.

3. The method according to claim 2, wherein the first separation utilizes no reducing agents.

4. The method according to claim 1, wherein the proteins in the mixture are purified in a second separation according to their molecular weight.

5. The method according to claim 4, wherein the second separation utilizes no more than about 2% SDS.

6. The method according to claim 4, wherein the second separation utilizes no more than about 1% SDS.

7. The method according to claim 4, wherein the second separation utilizes no more than about 0.1% SDS.

8. The method according to claim 1, wherein the purified proteins are assayed for NAD reductase activity.

9. The method according to claim 1, wherein the purified proteins are assayed for protein kinase activity.

10. The method according to claim 1, wherein the protein mixture is obtained from healthy cells.

11. The method according to claim 1, wherein the protein mixture is obtained from diseased cells.

12. The method according to claim 1, further comprising quantifying the purified proteins.

13. The method according to claim 1, further comprising identifying the purified proteins.

14. The method according to claim 11, wherein the purified proteins are identified by protein microsequencing.

15. The method according to claim 11, wherein the purified proteins are identified by mass spectrometry.

16. A system for purifying and characterizing proteins from a mixture comprising:
a separating apparatus that performs at least two orthogonal separations under conditions that preserve protein activity; and
a protein elution plate.

17. The system according to claim 16, wherein the separating apparatus comprises an IPG (immobilized pH gradient) strip.

18. The system according to claim 16, wherein the separating apparatus further comprises a polyacrylamide electrophoresis gel.

19. The system according to claim 16, wherein the system utilizes no reducing agents.

20. The system according to claim 16, wherein the system utilizes no more than about 2% SDS.

21. The system according to claim 16, wherein the system utilizes no more than about 1% SDS.

22. The system according to claim 16, wherein the system utilizes no more than about 0.1% SDS.

23. The system according to claim 16, wherein the protein elution plate has a plurality of receiving wells.

24. The system according to claim 16, wherein the protein elution plate has 1,536 receiving wells.

25. The system according to claim 16, wherein the protein elution plate comprises polypropylene.

26. The system according to claim 16, wherein the protein elution plate further comprises a semi-permeable membrane.

27. The system according to claim 26, wherein the semi-permeable membrane is attached to the protein elution plate through a gel.

28. The system according to claim 26, wherein the semi-permeable membrane comprises polyethersulfone or polyamide polymer.
